(19) United States
(12) Patent Application Publication
(10) Pub. No.: US 2011/0087103 A1
(43) Pub. Date: Apr. 14, 2011

(54) ULTRASOUND FOCUS POSITIONING DEVICE

(76) Inventor: Patrick Gross, Langensendelbach (DE)

(21) Appl. No.: 12/904,283

(22) Filed: Oct. 14, 2010

(30) Foreign Application Priority Data
Oct. 14, 2009 (DE) 10 2009 049 403.0

(51) Int. Cl. A61B 8/14

(52) U.S. Cl. 600/444

(57) ABSTRACT

A positioning device has a computer and an ultrasound device that is provided to expose biological tissue with high-intensity, focused ultrasound radiation, and an ultrasound transducer unit, a mechanical focusing unit and an electronic focusing unit for positioning and/or focusing of the high-intensity, focused ultrasound radiation of the ultrasound transducer unit. The computer is designed to adjust the focus position of the ultrasound transducer unit along at least one dimension by operating the mechanical focusing unit; and the computer is designed to regulate the focus position of the ultrasound transducer unit along the at least one dimension by operating the electronic focusing unit after the positioning by the mechanical focusing unit.

### BACKGROUND OF THE INVENTION

1. Field of the Invention

The invention comes from a positioning device and a positioning method for a positioning of a focus of a high-intensity, focused ultrasound beam of an ultrasound device.

2. Description of the Prior Art

A high-intensity, focused ultrasound beam is used to treat tumors in human tissue. The high-intensity, focused ultrasound beam is radiated into the body of a patient in the form of ultrasound pulses. The tumor tissue toward which the focus of the high-intensity, focused ultrasound beam is directed is thereby heated by the high-intensity, focused ultrasound beam. The heating has the effect that the tumor tissue is at least damaged or destroyed.

From U.S. Pat. No. 5,938,600 a device is known with an ultrasound device that is provided to expose biological tissue with high-intensity, focused ultrasound radiation. The ultrasound device has an ultrasound transducer unit, a mechanical focusing unit and an electronic focusing unit to position and/or focus ultrasound radiation of the ultrasound transducer unit. A focusing of the high-intensity, focused ultrasound radiation along one dimension ensues either mechanically by means of the mechanical focusing unit or electronically by means of the electronic focusing unit.

### SUMMARY OF THE INVENTION

An object of the present invention is to provide a positioning device that combines the advantages of mechanical adjustment and the advantages of electronic adjustment of a focus of high-intensity, focused ultrasound radiation in one adjustment step.

The invention includes a positioning device with a computer and an ultrasound device that is provided to expose biological tissue with high-intensity, focused ultrasound radiation and that has an ultrasound transducer unit, a mechanical focusing unit and an electronic focusing unit for positioning and/or focusing of the high-intensity, focused ultrasound radiation of the ultrasound transducer unit; and the computer is designed to adjust the focus position of the ultrasound transducer unit along at least one dimension by means of the mechanical focusing unit.

The computer is provided to regulate the focus position of the ultrasound transducer unit along the at least one dimension by means of the electronic focusing unit after positioning by means of the mechanical focusing unit. In this context, “provided” means specifically designed and/or specifically programmed or configured. The computer—which is formed by a processor and/or a regulation unit, for example—is advantageously designed to implement a first positioning by means of the mechanical focusing unit and to subsequently implement a fine adjustment by means of the electronic focusing unit along each adjustable dimension or along each adjustable degree of freedom. In particular, five degrees of freedom (three translation degrees of freedom and two rotation degrees of freedom) can be adjusted. For example, the mechanical focusing unit can be formed at least in part by a mechanical rail unit and/or a hydraulic unit and/or additional units that appear reasonable to those skilled in the art for mechanical positioning of the ultrasound transducer unit. In the embodiment according to the invention, a fast coarse adjustment over a large range can be achieved by means of the mechanical focusing unit and a fast (and in particular dynamic) fine adjustment or position regulation of the focus position can be achieved by means of the electronic focusing unit. Time-consuming adjustments via the mechanical focusing unit for an exact focus position—for example as are caused due to the mass inertia of the ultrasound transducer unit and/or due to the viscosity of the coupling medium accommodating the ultrasound transducer unit etc.—can be prevented by means of the electronic focusing unit. By means of the mechanical focusing unit, a broad range can additionally be used for a focus adjustment and the electronic focusing unit (whose electronic positioning of the focus is limited to a small region) can be limited to a fine adjustment of the focus position that compensates for inaccuracies of the mechanical positioning relative to a desired value.

Furthermore, the positioning device can have at least one position sensor that is designed to detect at least one characteristic spatial variable of the ultrasound transducer unit. The position sensor can be formed by a laser sensor, a mechanical sensor (for example a strain gauge) and particularly advantageously by an optical sensor. The position of the ultrasound transducer unit can be determined quickly from the characteristic spatial variable by means of the computer, and a time-saving fine adjustment by means of the electronic focusing unit can subsequently be achieved for an optimally exact focus adjustment of the ultrasound transducer unit. A focus position of the ultrasound transducer unit can additionally be determined together with a characteristic focus variable of the electronic focusing unit.

The positioning device can have a focus sensor that is designed to detect at least one characteristic spatial variable of the focus position, so the focus position of the high-intensity, focused ultrasound radiation can advantageously be detected directly and—insofar as necessary—a focus adjustment can be corrected quickly. The focus sensor can be formed by a magnetic resonance sensor, an ultrasound sensor, a computer tomography device and/or additional sensors that appear reasonable to those skilled in the art.

An advantageous adaptation of the focus position of the high-intensity, focused ultrasound radiation to a desired value—for example a position of a tumor—can be achieved if the positioning device possesses a target sensor that is provided to detect at least one characteristic spatial variable of a target region. In particular, a moving target region (for example a tumor that moves along with a breathing motion) can be detected with positional accuracy for a treatment. The target sensor can be formed by a magnetic resonance sensor, an ultrasound sensor, a computed tomography device, a camera, a respiration belt and/or additional sensors that appear reasonable to those skilled in the art.

Furthermore, the invention concerns a positioning method for positioning the focus of a high-intensity, focused ultrasound radiation of an ultrasound device to expose biological tissue, including the following steps: determine a desired value along at least one dimension; position an ultrasound transducer unit along the at least one dimension depending on the determined desired value to adjust a focus position by means of a mechanical focusing unit; detect the set focus position and compare between the desired value and the detected focus position by means of a computer.

In a correction step, a difference between the desired value and the mechanically adjusted focus position is at least partially corrected along the at least one dimension by means of an electronic focusing unit, so a fast and in particular dynamic focus adjustment can advantageously be achieved by a coarse adjustment over a large range by means of the mechanical focusing unit and a time-saving and exact fine adjustment by means of the electronic focusing unit. In particular, time-consuming adjustments by the mechanical focusing unit—for example based by a mass inertia of the ultrasound transducer unit and/or due to a viscosity of a coupling medium accommodating the ultrasound transducer unit etc.—can be avoided by means of the electronic focusing unit for an exact focus position. A wide range can additionally be used for a focus adjustment by means of the mechanical focusing unit and the electronic focusing unit—whose electronic positioning of the focus is limited to a small range—can be limited to a fine adjustment of the focus positioning that compensates for the imprecision of a mechanical positioning relative to a desired value. As used herein, a “desired value” means a position value of a target region and/or tumor toward which the ultrasound radiation should be directed.

In the focusing step the focus position is advantageously adapted by means of the mechanical and/or electronic regulation to a modification of the desired value, so a fast updating (tracking) and/or regulation of the focus position can be achieved, for example given an unwanted and/or unexpected change of the desired value.

Furthermore, in the focusing step the focus position of the high-intensity, focused ultrasound radiation can be moved continuously, at least in part with a movement of the desired value. For example, a movement of the desired value is triggered by a breathing of the patient. An advantageous treatment of a tumor can advantageously be achieved by the focus position at least partially following a movement of the desired value (and therefore of the tumor). In particular, the required energy radiation to damage or destroy tumor cells through a long radiation time can be achieved, and unwanted exposure of biological tissue surrounding the tumor can be prevented.

Furthermore, in the focusing step the difference between the desired value and the measured focus position can be at least partially corrected along the at least one dimension by means of the mechanical focusing unit, so corrections that are outside of the adjustment range of the electronic focusing unit can be made.

If, in the positioning step, the mechanical adjustment of the focus position is adapted to the desired value along the at least one dimension, which adaptation is essentially independent of the electronic focusing, an advantageous mechanical regulation of the positioning that is oriented on the respective desired value can be achieved. The electronic regulation of the focusing can additionally be limited to a correction of the mechanical regulation, such that a fast focusing over a wide region can be provided.

At least one characteristic spatial variable of the ultrasound transducer unit along the at least one dimension can be detected by means of a position sensor in a measurement step. The position of the ultrasound transducer unit can be determined from the characteristic spatial variable by the computer, and a fast fine adjustment by the electronic focusing unit can be subsequently achieved for an optimally exact focus adjustment of the ultrasound transducer unit.

In a correction step at least one parameter of a movement model is advantageously changed, so an advantageous adaptation of the desired value to a current position of the tissue to be exposed—for example due to a movement of the patient—can be achieved. A movement progression of the tissue to be exposed can be determined by means of the movement model using known and/or detected movement variables.

### BRIEF DESCRIPTION OF THE DRAWINGS

FIG. 1 shows a positioning device according to the invention in a schematic representation.

FIG. 2 shows a mechanical focusing unit in a schematic representation.

FIG. 3 is a workflow diagram of a positioning method according to the invention.

FIG. 4 is a diagram of an adjustment of a desired value at rest.

FIG. 5 is a diagram of an adjustment of a moving desired value.

### DESCRIPTION OF THE PREFERRED EMBODIMENTS

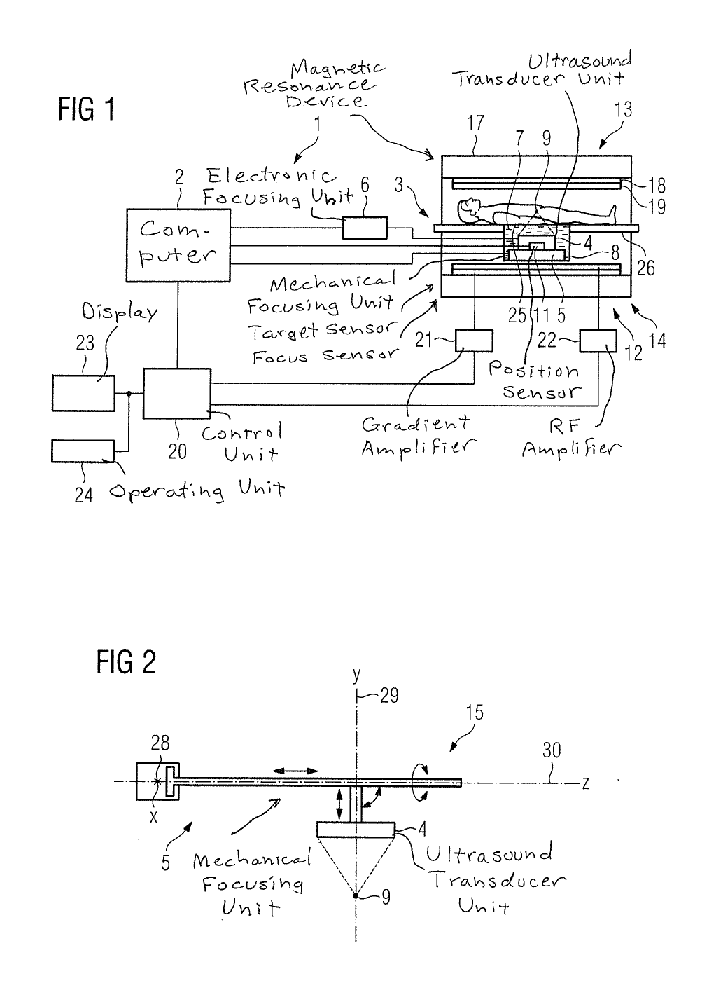

A positioning device 1 according to the invention is shown in FIG. 1. The positioning device 1 has a computer 2 and an ultrasound device 3. In the operation of the positioning device 1 the ultrasound device 3 is provided to expose biological tissue (for example a tumor in a patient) with high-intensity, focused ultrasound radiation. For this the ultrasound device 3 has an ultrasound transducer unit 4, a mechanical focusing unit 5 and an electronic focusing unit 6. The ultrasound transducer unit 4 and the mechanical focusing unit 5 are surrounded by an ultrasound contact fluid 7 and arranged within a housing 8 of the ultrasound device 3.

The computer 2 is designed to regulate a focus position 9 of the high-intensity, focused ultrasound radiation of the ultrasound transducer unit 4 by means of the mechanical focusing unit 5 and the electronic focusing unit 6 in order to specifically concentrate the high-intensity, focused ultrasound radiation onto a point or small region, and with this to achieve a desired energy injection (irradiation) for tumor treatment. The computer 2 is connected with the individual components and units of the positioning device 1 via a data line with which data and/or control signals can be exchanged during the operation of the positioning device 1.

The mechanical focusing unit 5 is designed for a positioning of the ultrasound transducer unit 4 in multiple dimensions, wherein in particular five degrees of freedom—three translation degrees of freedom and two rotation degrees of freedom—can be adjusted. This is realized in the present exemplary embodiment by means of a mechanical rail system 15 as this is schematically presented in FIG. 2, wherein alternative mechanical adjustment systems to adjust the five degrees of freedom of the ultrasound transducer unit 4 are conceivable at all times.

The ultrasound transducer unit 4 has a transducer array. The transducer array is composed of a number of transducers whose radiated ultrasound beams advantageously superimpose and form a common focus position 9. The individual transducers are individually activated by the computer 2 by the electronic focusing unit 6 during the operation of the positioning device 1.

Furthermore, the positioning device 1 has a position sensor 11 that is designed for a fast detection of a characteristic spatial variable of the ultrasound transducer unit 4. The position sensor 11 is formed by an optical sensor and arranged in a region of the ultrasound transducer unit 4. The positioning device 1 also has a target sensor 12 that is formed by a magnetic resonance device 13 and a focus sensor 14 that is likewise formed by the magnetic resonance device 13. The magnetic resonance device 13 is thus designed to detect a characteristic spatial variable of the focus of the high-intensity, focused ultrasound radiation and to detect a characteristic spatial variable of a target region, for example a tumor (FIG. 1).

The magnetic resonance device 13 has a magnet 17 to generate a strong and constant magnetic field. Furthermore, the magnetic resonance device 13 has gradient coils 18 that are provided to generate a linear gradient field and radio-frequency coils 19. The gradient coils 18 are controlled by a control unit 20 of the magnetic resonance device 13 via a gradient amplifier 21. The radio-frequency coils 19 are controlled by the control unit 20 via a radio-frequency (RF) amplifier 22 and are additionally provided to receive a magnetic resonance signal. The magnetic resonance device 13 also has a monitor display 23 by means of which magnetic resonance exposures and/or results evaluated by control unit 20 can be presented. Adjustments and/or parameter changes by an operator can be made by an operating unit 24 of the magnetic resonance device 13 (FIG. 1).

The ultrasound device 3 is arranged on the underside 25 of a patient bed 26 of the magnetic resonance device 13. The patient bed 26 has a membrane permeable to ultrasound radiation in the region of the ultrasound device 3. The ultrasound device 3 rests directly on the underside 25 of the membrane of the patient bed 26 in order to minimize ultrasound radiation-reflecting transitions between the ultrasound transducer unit 4 and the patient. The ultrasound device 3 and the position sensor 11 are fashioned so as to be magnetically compatible for an interference-free operation of the positioning device 1.

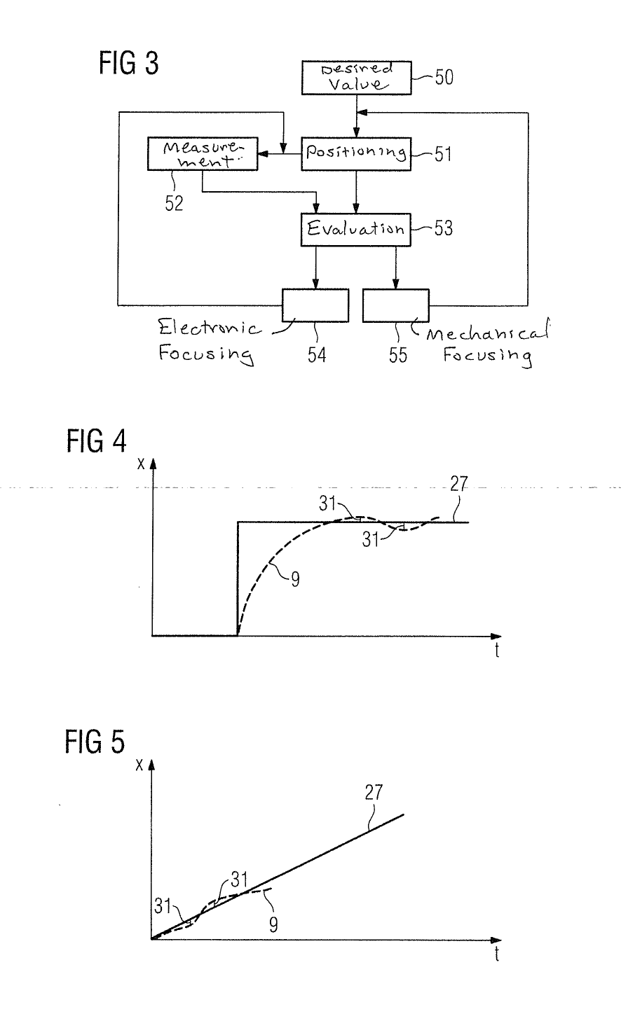

In the operation of the positioning device 1, a positioning method is started to position the focus of the high-intensity, focused ultrasound radiation (FIG. 3). A desired value 27 or multiple desired values 27 is/are hereby initially determined in a desired value step 50. The desired value 27 or desired values 27 can be determined along at least one franking imprint or one adjustable degree of freedom or along all five degrees of freedom. The desired values 27 can be input manually by means of the operating unit 24 and/or be read out from a data storage (not shown in detail) of the positioning device 1 and/or be currently determined by a desired value measurement. The desired value measurement ensues by acquisition of magnetic resonance exposures by means of the magnetic resonance device 13.

After the measurement and/or input of the desired values 27, these are conducted to the computer 2 in the desired value step 50 and a time and/or spatial progression of the individual desired values 27 is determined there for an adjustment of focus positions 9 of the ultrasound transducer unit 4. The time and/or spatial progression of the individual desired values 27 is dependent on a position of the tissue to be exposed within the patient, such that the progression is determined with the aid of a movement model of the patient given moving desired values 27 (as these are present in tumors that move along with a breathing motion, for example). Furthermore, differences in the anatomy of the patient can also enter into the determination of the progression of the desired values 27.

In a positioning step 51 the ultrasound transducer unit 4 is subsequently positioned by means of the mechanical focusing unit 5 along the adjustable dimensions or the adjustable degrees of freedom depending on the desired values 17 to adjust the focus positions 9. This ensues along an x-axis 28, a y-axis 29 and a z-axis 30 by means of the mechanical rail system 15 of the mechanical focusing unit 5. The ultrasound transducer unit 4 is additionally borne such that it can rotate around the z-axis 30 and around the x-axis 28, such that a more effective radiation angle for the high-intensity, focused ultrasound radiation is achieved by means of the mechanical focusing unit 5 (FIG. 2).

A measurement step 52 is started by the computer in parallel with the positioning step 51. The adjusted focus position 9 is detected in the measurement step, such that measurement results of the adjusted focus position 9 already exist at least in part after the end of the positioning step 51. The adjusted focus position 9 is detected by means of the position sensor 11. This directly and quickly detects characteristic spatial variables of the ultrasound transducer unit 4 along the five adjustable degrees of freedom. The characteristic spatial variables are relayed to the computer 2 so that an exact position of the ultrasound transducer unit 4 is determined by the computer 2 using the measured characteristic spatial variables. A current focus position 9 of the high-intensity, focused ultrasound radiation is calculated by the computer 2 together with the current focusing parameters of the ultrasound transducer unit 4, for example a phase and/or an amplitude of the ultrasound radiation. The current focus position 9 can additionally be determined by means of the magnetic resonance device 13 in that an energy injection into the focus position 9 is detected in the magnetic resonance exposures using a heating profile.

Furthermore, a position of the desired value 27 can be determined in the measurement step 52, as this is in particular reasonable given desired values 27 or tumors that move. However, a calibration of the magnetic resonance device 13 for the individual measurements is hereby required, in particular measurement of the desired value 27 and measurement of the focus position 9.

In an alternative embodiment of the positioning method, the positioning step 51 can also proceed chronologically before the measurement step 52.

In an evaluation step 53, the desired value 27 is subsequently compared by the computer 2 with the detected focus position 9 and a difference between the two positions is determined. A possible difference between the two positions can result from the mechanical focusing unit 5 in that this reacts with a delay (for example due to a viscosity and/or mass inertia of the ultrasound contact fluid) and therefore a desired focus position 9 is reached with a delay. A time-limited activation rate and/or a limited torque of motors of the mechanical focusing unit 5 can also additionally contribute to a difference of the two positions. An unforeseen movement of the patient can also lead to additional differences etc.

In order to at least partially destroy a tumor, a predetermined energy injection is required. This is achieved if high-intensity, focused ultrasound radiation is focused at a desired value 27 in the tumor over a predetermined time period. For this, after the evaluation step 53 a difference between the desired value 27 and the mechanically adjusted focus position 9 of the high-intensity, focused ultrasound radiation is at least partially corrected along the mechanically adjusted dimensions and/or the mechanically adjusted degrees of freedom by the computer 2 in a focusing step 54 by means of the electronic focusing unit 6. In FIG. 4 this is schematically represented in a plot—along the dimension x over time t. Due to the delayed reaction of the mechanical focusing unit 5, the mechanically adjusted focus position 9 achieves the moving desired value only imprecisely and with a delay. In the evaluation step 53 a correction value 31 that is set via the electronic focusing unit 6 is determined by means of the computer 2. For this purpose, control signals are generated by the computer 2 and relayed to the electronic focusing unit 6. For example, in the electronic focusing unit 6 an amplitude and/or a phase of the ultrasound radiation are thereupon adapted for the individual transducers of the ultrasound transducer unit 4 and relayed to the individual transducers, and the focus position 9 is adjusted to the desired value 27.

After an adjustment of the focus position 9 has occurred, either a treatment by means of the ultrasound device 3 can be started or the focus position 9 and/or the desired value 27 can be checked again by resuming the method at the measurement step 52. Insomuch as a difference between the focus position 9 and the desired value continues to be established in the evaluation step 53, a readjustment of the focus position 9 by the computer 2 ensues by means of the electronic focusing unit 6.

If, after the evaluation step 53, large differences exist between the two positions that are arranged outside of a regulation range of the electronic focusing unit 6, in the focusing step 54 the difference between the desired value 27 and the measured focus position 9 is at least partially corrected by means of the mechanical focusing unit 5, wherein the mechanical focusing in the focusing step 54 ensues essentially analogously to the steps for electronic focusing. An electronic fine adjustment of the focus position 9 by means of the electronic focusing unit 6 according to the procedure described above subsequently ensues in the focusing step 54.

Given desired values 27 or tumors at rest, a step-by-step scanning of the tumor by means of the ultrasound transducer unit 4 is provided along the individual desired values 27. The focus position 9 adjusted to desired value 27 will hereby maintain a predetermined exposure time, such that the required energy injection at the desired value 27 is achieved. The focus position 9 is subsequently adjusted to the next desired value 27. An adjustment of the focus position 9 according to the positioning method described above ensues for each of the desired values 27. Alternatively, a continuous progression of the ultrasound transducer unit 4 in the scanning of the tumor is conceivable at any time.

The mechanical positioning and/or regulation of the focus of the ultrasound transducer unit 4 in the positioning step 51 ensues under consideration of the predetermined desired value 27 and is essentially independent of the electronic focusing at the desired value 27 by means of the electronic focusing unit 6 along the adjustable dimensions and/or the adjustable degrees of freedom.

In contrast to this, given moving or varying desired values 27 or tumors the computer 2 is provided to adapt the mechanical and/or electronic regulation of the focus position 9 in the focusing step 54 to the variation of the desired value and to make a new adjustment of the focus position 9 to the modified desired value 27. Given moving desired values 27 that move along a movement model, in the focusing step 54 the focus position 9 is regulated at least partially with the movement of the desired value so that the focus position 9 is moved continuously with the desired value 27. In FIG. 5 this is represented schematically in a plot along the dimension x over the time t. It can additionally be provided that, given moving desired values 27 or tumors, an energy injection by means of the ultrasound transducer unit 4 ensues only during a partial range of a repeating movement sequence in order to achieve an effective injection angle and/or an effective injection time. The focus position 9 is hereby moved with the desired value 27 only in the partial range. A regulation or control of the continuous associated movement of the focus position 9 of the ultrasound transducer unit 4 by the computer 2 ensues via the mechanical focusing unit 5 and the electronic focusing unit 6.

In exceptional cases in which a variation of the desired value 27 that deviates significantly from the movement model and/or a mechanical and/or electronic regulation of the focus position that has already occurred has been unsuccessful is established by the computer 2 in the evaluation step 53, a correction step 55 is started the computer 2. The correction step 55 is started instead of the focusing step 54. In the correction step 55 a change of at least one parameter of the movement model is provided. A correction for the chronological or spatial sequence of the individual desired values 27 for the adjustment of focus positions 9 of the ultrasound transducer unit 4 is also determined by the computer 2 with the correction in the movement model. The positioning method is subsequently resumed at the positioning step 51.

Although modifications and changes may be suggested by those skilled in the art, it is the intention of the inventor to embody within the patent warranted hereon all changes and modifications as reasonably and properly come within the scope of his contribution to the art.

I claim as my invention:

1. A positioning device for positioning an ultrasound focus, comprising:
an ultrasound device that emits high-intensity, focused ultrasound from a focus in the ultrasound device;
a mechanical focusing unit that mechanically interacts with said focus;
an electronic focusing unit that interacts with said focus; and
a computer connected to said mechanical focusing unit and to said electronic focusing unit, said computer being configured to operate said mechanical focusing unit to initially mechanically adjust a position of said focus along at least one dimension and to subsequently operate said electronic focusing unit to regulate the position of said focus along said at least one dimension after adjusting the position of the focus with said mechanical focusing unit.

2. A positioning device as claimed in claim 1 comprising at least one position sensor that detects at least one characteristic spatial variable associated with emission of said ultrasound radiation by said ultrasound transducer unit, said at least one position sensor emitting a sensor output to said computer and said computer being configured to use said sensor output to operate at least one of said mechanical focusing unit and said electronic focusing unit.

3. A positioning device as claimed in claim 2 wherein said at least one position sensor is a focus sensor that detects at least one characteristic spatial variable of said position of said focus.

4. A positioning device as claimed in claim 1 wherein said ultrasound device is configured to emit said high-intensity focused ultrasound radiation at a target region, and wherein said positioning device comprises a target sensor that detects at least one characteristic spatial variable of said target region, said target sensor emitting a target sensor output to said computer and said computer being configured to use said target sensor output to operate at least one of said mechanical focusing unit and said electronic focusing unit.

5. A method for positioning a focus of a high-intensity focused ultrasound radiation emitter, comprising the steps of:
determining a desired value along at least one dimension of a focus in said ultrasound emitter from which said high-intensity, focused ultrasound radiation is emitted;
from a computer, operating a mechanical focusing unit to mechanically adjust a position of said focus along said at least one dimension to set said focus to a set focus position;
detecting said set focus position;
supplying said desired value and the detected set focus position to said computer and, in said computer, comparing said desired value and the detected set focus position to identify a difference there between; and
from said computer, operating an electronic focusing unit to electronically further adjust the position of said focus along said at least one dimension dependent on said difference.

6. A positioning method as claimed in claim 5 comprising, from said computer, operating at least one of said mechanical focusing unit and said electronic focusing unit to regulate said position of said focus dependent on said desired value.

7. A positioning method as claimed in claim 5 wherein said desired value exhibits movement, and comprising, from said computer, operating said electronic focusing unit to electronically continuously move the position of said focus to at least substantially correspond to said movement of said desired value.

8. A positioning method as claimed in claim 5 comprising, from said computer, also operating said mechanical focusing unit to mechanically adjust the position of said focus dependent on said difference.

9. A positioning method as claimed in claim 5 comprising, from said computer, operating said mechanical focusing unit to mechanically adjust the position of said focus with respect to said desired value along said at least one dimension independently of electronically adjusting the position of the focus by operating said electronic focusing unit.

10. A positioning method as claimed in claim 5 comprising detecting at least one characteristic spatial variable associated with said high intensity focused ultrasound radiation and providing said at least one spatial variable to said computer and, from said computer, operating at least one of said mechanical focusing unit and said electronic focusing unit dependent on said characteristic spatial variable.

11. A positioning method as claimed in claim 5 comprising providing said computer with a movement model representing expected movement of said desired value, and, from said computer, operating at least one of said mechanical focusing unit and said electronic focusing unit by changing at least one parameter of said model.
